(19) United States
(12) Patent Application Publication
(10) Pub. No.: US 2017/0333532 A1
(43) Pub. Date: Nov. 23, 2017

(54) ADP'ASE-ENHANCED APYRASE THERAPY FOR WOUNDS, MICROBIAL INFECTION, SEPSIS, AND HETEROTOPIC OSSIFICATION

(71) Applicant: APT THERAPEUTICS, INC, Saint Louis, MO (US)

(72) Inventors: Ridong CHEN, Naperville, IL (US); JEONG Seog Soon, Naperville, IL (US)

(73) Assignee: APT THERAPEUTICS, INC, Saint Louis, MO (US)

(21) Appl. No.: 15/522,766

(22) PCT Filed: Oct. 29, 2015
(86) PCT No.: PCT/US15/57910

§ 371 Date: Apr. 27, 2017

Related U.S. Application Data
(60) Provisional application No. 62/069,997, filed on Oct. 29, 2014.

(51) Int. Cl. A61K 38/46; A61K 38/12

(52) U.S. Cl. CPC A61K 38/46; A61K 38/12; C12Y 306/01005

(57) ABSTRACT

This invention provides composition and methods of treating subjects with microbial infection, sepsis, wounds, heterotopic ossification, or combination thereof. In each case, the treatment methods of the present invention comprise administering ADPase-enhanced apyrase agents, alone or in combination with an antimicrobial.

### CROSS REFERENCE TO RELATED APPLICATIONS

This application claims the benefit of U.S. Provisional Patent Application No. 62/069,997 filed 29 Oct. 2014.

### TECHNICAL FIELD

The present invention relates to Apyrase Agents, methods for treating subjects with microbial infection, sepsis, wound or burn, and heterotopic ossification thereof.

### BACKGROUND

Wound healing is a complex process that requires a highly regulated series of events including inflammation, tissue formation, revascularization and tissue remodeling. However, this is impaired in certain pathological conditions such as thrombosis, ischemia, diabetics and infection.

Open injuries have a potential for at least three serious outcomes: bacterial infection (especially antibiotic resistant infections), heterotopic ossification, and sepsis.

Serious bacterial wound infections (including gas gangrene and tetanus) which can in turn lead to long term disabilities, chronic wound or bone infection, and death.

Wound infections also affect military operations, homeland preparedness against natural disasters, and preparedness against terrorism attacks.

In both general public and wartime or natural disaster emergency wound care, effective firsthand measures are required to prevent and treat wound infections—failure of which could result in the development of chronic wound infections that are difficult to eradicate.

Treatment of wounds with antimicrobials has long been the standard of care. The term “antibiotic” is broadly defined as a chemical compound produced by one microorganism that inhibits the growth of a different microorganism. Today, there are more than 150 antibiotics classified by their chemical structures and mechanisms of action.

Through the abuse and misuse of antibiotics, many bacteria have developed resistance to these antibiotics which has become a worldwide problem. Studies over the past decades have documented the extensive human and economic toll of antibiotic resistance. Each year in the United States, at least 2 million people acquire serious infections with antibiotic resistant bacteria. Recent studies show that methicillin-resistant Staphylococcus aureus (MRSA) alone caused 250,000-300,000 hospital acquired infections, about 2.7 million hospital days, and 12,000 deaths annually, resulting in annual costs of about $20 billion in the early 2008 and that vancomycin-resistant enterococci (VRE) caused about 26,000 infections in US hospitals in 2004. Resistance to antibiotics refers to a type of drug resistance where a microorganism is able to survive exposure to an antibiotic intended to inhibit the growth of the microorganism or to kill it. Antibiotic resistance may arise by spontaneous or induced genetic mutation in a microorganism.

Resistance is likely resulting from widespread antibiotic use in human medicine, veterinary medicine, agriculture, and livestock production. Any use of antibiotics can increase selective pressure in a population of bacteria to allow the resistant bacteria to thrive and the susceptible bacteria to die off. As resistance towards antibiotics becomes more common, a greater need for alternative treatments arises. However, despite a push for new antibiotic therapies there has been a continued decline in the number of newly approved drugs. Thus, antibiotic resistance poses a significant problem. There is a crucial need for novel antimicrobials that effectively inhibit the growth of infectious and pathogenic microbes as well as for novel strategies for improving the effectiveness of extant antimicrobials.

Heterotopic ossification (HO) is a musculoskeletal disease that is characterized by the formation of mature bone in soft tissues such as muscle, tendon, or fascia and is a frequent complication following trauma, burns, upper extremity injuries, or surgeries. Over 60% of severe burn patients will develop HO in at least one joint during recovery. Additionally, HO can occur secondary to other conditions, including soft-tissue trauma, amputation, central nervous system injury (e.g., traumatic brain injuries, spinal cord lesions, tumors, encephalitis), vasculopathies, arthroplasties (e.g., total hip arthroplasty), and end-stage cardiac valve disease.

The pathogenesis of heterotopic ossification remains unclear, though the inciting event is thought to be inflammation caused by trauma, surgery, or burns. This inflammation stimulates the recruitment of mesenchymal stem cells (MSCs) as well as endochondral ossification of resident MSCs. Current treatment and prophylactic strategies are not effective to prevent HO formation and/or to treat and reconstruct joints once HO has developed. Conventional technologies for treatment involve surgical extirpation of the heterotopic bone. However, even after a technically successful operation, over 75% of patients have difficulty maintaining their range of motion, 35% of patients have residual bone, over 10% of patients recur, and even those patients without complications suffer from significant joint contractures.

Sepsis is a major cause of death worldwide. Approximately 750,000 cases of sepsis occur annually in the USA, with a mortality rate of 28.6%. The key event underlying this life-threatening complication is the overwhelming inflammatory host response to the infectious agent leading to widespread microvascular thrombosis, ischemia, and multiple organ dysfunction syndrome.

In the face of the substantial impact, cost, and morbidity and mortality of these three consequences of wounds (i.e. bacterial infection, HO, and sepsis), a substantial need exists for the development of treatment methods.

### SUMMARY OF THE INVENTION

This invention provides compositions and methods of treating wounds with an ADPase enhanced apyrase (“Apyrase Agent”), alone or in combination with an antimicrobial. In one embodiment the antimicrobial is colistin or an antibacterial polymer (e.g. E2 or E4).

In another aspect, this invention provides compositions and methods of treating heterotopic ossification with Apyrase Agent, alone or in combination with an antimicrobial.

In one aspect, this invention provides compositions and methods of treating sepsis, severe sepsis, or septic shock with Apyrase Agent, alone or in combination with an antimicrobial.

In another aspect, Apyrase Agent (alone or in combination) increases the effectiveness of an antimicrobial.

In some embodiments the combination treatment comprising administering a first composition comprising the apyrase and administering a second composition comprising the antimicrobial. In other embodiments, the combination treatment comprising administering a first composition comprising the antimicrobial and a second composition comprising the Apyrase Agent. In other embodiments, the combination treatment comprising co-administering the antimicrobial and the Apyrase Agent.

Some embodiments provide methods further comprising testing the subject for an infection. In some embodiments, the testing is before the administering and in some embodiments the testing is after the administering. In some embodiments, a second treatment (e.g., of the Apyrase Agent and an antimicrobial) is administered to the subject. For example, some embodiments provide that a dosage of the second administering is determined based on a result of a testing.

### BRIEF DESCRIPTION OF THE DRAWINGS

FIG. 1 depicts the effect of colistin and various volumes of placebo on the growth curve of E. coli K-12.

FIG. 2 depicts the effect of Apyrase Agent at various concentrations on the growth curve of E. coli K-12.

FIG. 3 depicts the effect of a combination of colistin and Apyrase Agent at various concentrations on the growth curve of E. coli K-12.

### DETAILED DESCRIPTION OF THE INVENTION

As used here, the following definitions and abbreviations apply.

“Antimicrobial” refers to agents that kill microorganisms or inhibits their growth. Antimicrobials include antibiotics, disinfectants, and antiseptics.

“Examplary” (or “e.g.” or “by example”) means a non-limiting example.

“HO” means heterotropic ossification.

“Topically” refers to application of the compositions of the present technology to the surface of the skin and mucosal cells and tissues (e.g., alveolar, buccal, lingual, masticatory, or nasal mucosa, and other tissues and cells which line hollow organs or body cavities).

“Treatment” (or “treat”) refers to prophylaxis or to a condition with clinically present symptoms. The use of “treatment” contemplates conditions of a wound that make the subject susceptible to microbial infection, HO, or sepsis. It also contemplates a subject with a wound having one or more of a microbial infection, HO, or sepsis. A treatment is successful if one or more of a microbial infection, HO, or sepsis is prevented in a subject at risk, or if the clinical course of one or more of a microbial infection, HO, or sepsis is altered (e.g. slowed progression or regression).

“Wound” refers broadly to injuries to tissue including the skin, subcutaneous tissue, muscle, bone, and other structures initiated in different ways, for example, surgery, (e.g., open post-cancer resection wounds, including but not limited to, removal of melanoma and breast cancer, etc.), contained post-operative surgical wounds, pressure sores (e.g., from extended bed rest), wounds induced by trauma, and burns. The term is not limited with regards to the cause, e.g., a physical cause such as bodily positioning (e.g., as in bed sores) or an impact as with trauma, a chemical process such as a burn or exposure to a caustic chemical substance, or a biological cause such as a disease process, an aging process, an obstetric process, or any other manner of biological process.

### Apyrase Agents

An Apyrase Agent useful according to the present invention is any apyrase that has ATPase and ADPase activities and where the ratio of ATPase to ADPase activity is less than 10:1 or optionally less than 8:1 or optionally less than 5:1 or optionally less than 4:1 or optionally less than 2:1 (units/mg compared to units/mg).

By ATPase activity, it is meant the activity that catalyzes the hydrolysis of phosphoanhydride bonds of adenosine triphosphate (ATP) to adenosine monophosphate (AMP) using two molecules of water (Reaction 1). By ADPase activity, it is meant the activity that catalyzes the hydrolysis of adenosine diphosphate (ADP) to AMP using one molecule of water (Reaction 2).

ATP+2·H₂O→AMP+2 phosphate. Reaction 1

ADP+H₂O→AMP+phosphate. Reaction 2

A Unit of Apyrase Agent activity is defined here as the amount of activity that will liberate 1.0 μmole of inorganic phosphate from 0.5 mM ATP per min at pH 7.4 at 37° C.

A Unit of ATPase activity is defined here as the amount of activity that will liberate 1.0 μmole of inorganic phosphate from 0.5 mM ATP per min at pH 7.4 at 37° C.

A Unit of ADPase activity is defined here as the amount of activity that will liberate 1.0 μmole of inorganic phosphate from 0.5 mM ATP per min at pH 7.4 at 37° C.

To determine the ratio of ADPase and ATPase activity, samples are incubated with 50 μM [¹⁴C]-ADP or [¹⁴C]-ATP in 96-well plates for 5 min. Subsequently, plates are placed on melting ice, and 10 μl “stop solution” (160 mM disodium EDTA, pH 7.0, 17 mM ADP, 0.15 M NaCl) is immediately added to each well. Nucleotides, nucleosides, and bases are separated by TLC. Radioactivity is quantitated by radio-TLC scanning, and results are calculated as averages of triplicate measurements following subtraction of buffer blanks.

Certain Apyrase Agents can also hydrolyze other nucleoside triposphates such as GTP, CTP, UTP, and other nucleoside diphosphates such as GDP, CDP, and UDP with various substrate specificities or preferences. The hydrolysis reactions catalyzed by Apyrase Agents require either calcium or magnesium as co-factor.

Apyrase Agents can belong to one of several well-known families of apyrases. For example, an Apyrase Agent can belong to the CD39 class. By way of example, an Apyrase Agent can be a CD39L class agent (e.g. L1-L8). The CD39 Apyrase Agent can be soluble, e.g. sol CD39 or sol CD39L3. Genera and species of useful agents are described by Chen et al. (U.S. Pat No. 7,247,300) and described by Jeong et al (U.S. Pat. No. 7,390,485). Additionally, Apyrase Agents include EN-apyrases taught in U.S. Pat. No. 8,771,683. Apyrase Agents include apyrase homologs.

An Apyrase Agent can be a soluble calcium-activated nucleotidase (SCAN gi 20270339; SCAN-1 gi: 22218108; EC 3.6.1.6) as described by Smith et al. (Arch. Biochem. Biophys., [2002], 406: 105-115). Such agents have sequence homology with the bed bug Cimex lectularius apyrase (gi: 4185746) (Valenzuela et al. J. Biol. Chem., [1998], 273:30583-305900).

An Apyrase Agent can be a 5′-nucleotidase (gi: 33520072; EC 3.1.3.5), for example, as found in humans. Such agents have sequence homology with the mosquito Aedes aegypti apyrase (gi: 1703351) (Champagne et al. Proc. Natl. Acad. Sci. USA, [1995], 92:694-698).

Useful Apyrase Agents can be an inositol polyphosphate 5′-phosphatase (gi: 346209; EC 3.1.3.56), for example, as occur in humans. Such agents have sequence homology with the Rhodnius prolixus apyrase (gi; 1546841) (Sarkis et al. Biochem. J., [1986], 233:885-891).

An Apyrase Agent can be an agent modified to increase the ATPase or ADPase activities, thereby increasing the therapeutic activity according to the present invention. Examples of such agents (or “homologs”) are sol CD39L3 R67G, sol CD39L3 R67G T69R (SEQ ID No:3 of US 2015/0265685 A1, without the first 6 N ter amino acid residues), and sol CD39L3 T69R, as taught in Chen et al. (U.S. Pat No. 7,247,300) and in Jeong et al (U.S. Pat. No. 7,390,485).

In one aspect, Apyrase Agent can be a human-derived apyrase.

In another embodiment, the Apyrase Agent is produced by a yeast expression system.

In another embodiment, the Apyrase Agent is produced by as a recombinant protein expressed in a mammalian cell line

In another embodiment, the Apyrase Agent is an engineered eucaryotic (e.g., mammalian) apyrase.

“Homologs”, in reference to Apyrase Agents, are agents that have (1) structural similarity to a class of apyrase (e.g. as discussed above, CD39; SCAN, SCAN-1, 5′-nucleotidase, inositol polyphosphate 5′-phosphatase); (2) ADPase and ATPase activity; and (3) one or more substitutions (i.e. differences) from wild type apyrase. By “structurally similar” it is meant about or more than about any of 80% or 90% or 95% homology. Moreover, conservative substitutions, as they are now commonly known in the art, are expressly contemplated.

### Antimicrobials

An antimicrobial can be, e.g. an antibiotic. An antibiotic, useful according to the present invention, can be any antibiotic.

Through insight of the inventors, colistin has been selected in the examples herein as representing a class of antibiotics and indeed, the results that can be obtained as asserted here can be obtained by other members of such a class. By way of example, colistin is a member of a polymyxin family of antibiotics. Colistin is a member of a group of antibiotics known as polycation antibiotics. Colistin is a member of a group of antibiotics known as peptide antibiotics. Colistin is a member of a group of antibiotics known as aminoglycoside antibiotics. The structural features of members of these antibiotics (i.e. aminoglycoside antibiotics, polycation antibiotics, or peptide antibiotics) allow them bind to lipopolysachharides (lps) of bacteria and alter packing arrangement, leading to death. Without being bound by theory, it is believed that this common structural and function feature serves a role in antibiotic action (bacterial killing) and antibiotic resistance and is the target for overcoming resistance by Apyrase Agent. Thus, resistance to colistin and other members of these groups of antibiotics are believed to be blunted or prevented by the Apyrase Agent according to the instant invention.

As used herein, “colistin-type” antibiotics include, e.g., alpha defensin (NP-1), bacitracin, bactenecin, Beta defensin 1, buforin II, cecropin A, cecropin P1, Colimycin, gentamycin, Gramicidin S, indolicidin, magainin II, nisin, polymxin B, ranalexin, tachyplesin, and tobramycin.

With the teaching herein, the skilled artisan should immediately appreciate that all references herein to “colistin” apply equally to “colistin-type” antibiotics.

Examples of other antibiotics useful according to the instant invention antimicrobial polymer E2, antimicrobial polymer E4, macrolides, polymyxins, penicillin, cephalosporin, carbepenem, monobactam, beta-lactam inhibitor, oxaline, aminoglycoside, chloramphenicol, sulfonamide, glycopeptide, quinolone, tetracycline, fusidic acid, trimethoprim, metronidazole, clindamycin, mupirocin, rifamycins, streptogramin, lipoprotein, polyene, azole, or echinocandin. Additional specific antimicrobials that find use within the scope of the technology are erythromycin, nafcillin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linezolid, temafloxacin, tosufloxacin, clinafloxacin, sulbactam, clavulanic acid, amphotericin B, fluconazole, itraconazole, ketoconazole, and nystatin.

### Therapeutic Compositions and Administration of Apyrase Agents

### Administration Routes

The present invention provides compositions comprising a biologically effective amount of Apyrase Agent or biologically active derivative in a pharmaceutically acceptable dosage. Therapeutic composition of Apyrase Agents or biologically active derivatives (i.e. homologs) may be administered clinically to a patient before symptoms, during symptoms, or after symptoms.

Administration of Apyrase Agents to achieve therapeutic effect may be given by oral, transdermal, intradermal, transmucosal, inhalation, subcutaneous, intramuscular, or parenteral administration. Parenteral administration can be, e.g., by intraveneous or intraperitoneal injection such as bolus injection, continuous infusion, sustained release, or other pharmaceutically acceptable techniques. Certain clinical situations may require administration of Apyrase Aagents as a single effective dose, or may be administered as multiple doses or multiple locations.

It should also be recognized by the skilled artisan that alternative formulations of Apyrase Agents can allow for alternate routes of administrations (e.g. oral [e.g. enteral, sublabial, or respiratory], ophthalmic, otologic, nasal, rectal, or dermal).

### Dosage Forms

Optionally Apyrase Agents are administered to patients in a pharmaceutically acceptable form containing physiologically acceptable carriers, excipients or diluents. Such diluents and excipients may be comprised of neutral buffered saline solution, antioxidants (for example ascorbic acid), low molecular weight polypeptides (for example polypeptides≦10 amino acids) amino acids, carbohydrates (for example, glucose, dextrose, sucrose, or dextrans), chelating agents such as EDTA, stabilizers (such as glutathione). Additionally, cosubstrates for the Apyrase Agents, for example, calcium (Ca²⁺) may be administered at time of dosage for maximal activity of the enzyme. Such carriers and diluents are nontoxic to the patient at recommended dosages and concentrations.

Instant compositions can be administered by implantation by inserting an implantable composition, e.g., microspheres, hydrogels, polymeric reservoirs, cholesterol matrixes, polymeric systems, matrix erosion and/or diffusion systems, compressed, fused, or partially-fused pellets.

Instant compositions can optionally be administered by means of a spray, tape, dry dressing, cleansing, film, or foam dressings.

Instant compositions can be administered by systemically by injection, e.g. of a composition that is encapsulated in liposomes.

The technology provided herein also includes kits for use in the instant methods. Kits of the technology comprise one or more containers comprising apyrase and an antimicrobial (e.g., in the same or in a different container).

### Dosing Frequency

In some embodiments, a single dose of a composition according to the technology is administered to a subject. In other embodiments, multiple doses are administered over two or more time points, separated by hours, days, weeks, etc. In some embodiments, compounds are administered over a long period of time (e.g., chronically), for example, for a period of months or years (e.g., 1, 2, 3, 4, 5, 6, 7, 8, 9, 10, 11, 12, or more months or years). In such embodiments, compounds may be taken on a regular scheduled basis (e.g., daily, weekly, etc.) for the duration of the extended period.

### Dosing

Dosage requirements of an Apyrase Agent may vary significantly depending on age, race, weight, height, gender, duration of treatment, methods of administration, biological activity of Apyrase Agents, and severity of condition or other clinical variables. Effective dosages may be determined by a skilled physician or other skilled medical personnel.

By way of example, dosing of Apyrase Agent by parenteral administration can vary between 10 units/kg and 1,000 units/kg of body weight. Optionally, dosing of Apyrase Agent by parenteral administration can vary between 50 units/kg and 300 units/kg of body weight.

By way of example, dosing of Apyrase Agent by topical administration can vary between 0.1 units/kg and 100 units/kg of body weight. Optionally, dosing of Apyrase Agent by topical administration can vary between 0.3 units/kg and 10 units/kg of body weight.

By way of example, dosing of Apyrase Agent by topical administration can also vary between 0.01 units/cm² of surface area treated and 10 units/cm² of surface area treated. Optionally, dosing of Apyrase Agent by topical administration can vary between 0.1 units/cm² of surface area treated and 1 units/cm² of surface area treated.

### Testing

In some embodiments, a subject is tested to assess the presence, the absence, or the level of a disease (e.g., an infection, sepsis, or heterotopic ossification), e.g., by assaying or measuring a biomarker, a metabolite, a physical symptom, an indication, etc., to determine the risk of or the presence of an infection, and thereafter the subject is treated with Apyrase Agent and (in some embodiments) an antimicrobial based on the outcome of the test. In some embodiments, a patient is tested, treated, and then tested again to monitor the response to therapy. In some embodiments, cycles of testing and treatment occur without limitation to the pattern of testing and treating (e.g., test/treat, test/treat/test, test/treat/test/treat, test/treat/test/treat/test, test/treat/treat/test/treat/treat, etc.), the periodicity, or the duration of the interval between each testing and treatment phase.

### Metabolic Modulation

Apyrase Agents, according to the present invention, have enhanced ADPase activity relative to ATPase activity in comparison to potato apyrase. Apyrase Agent's unexpectedly superior action is due, in part, to its enhanced ADPase activity. Without being bound by theory, it is believed that this superior action is due to a depletion of extracellular ATP (eATP) and ADP (eADP) and generation of extracellular adenosine (eADO) at the sight of the injury.

### Wound Repair.

For example, it is known that extracellular ATP levels increase during tissue injury, infection, hypoxia, and inflammatory conditions, due to release from resident and infiltrating cells. Released eATP binds to P2X7 or P2Y2 receptors that activate endothelial cells, monocytes, and lymphocytes to secrete proinflammtory cytokines and induces immune responses. Also, even at low concentrations, ATP induces the migration and differentiation of dendritic cells. Apyrase Agent treatment (alone or in combination with an antimicrobial), according to the present invention, results in a decreases eATP at injury sights.

It is also known that eADP plays a central role in activating and recruiting platelets via binding to P2Y₁ and P2Y₁₂ receptors, causing thrombosis and inflammation. It is believed by the inventor that ATPase activity and ADPase activity of Apyrase Agent following administration according to the present invention, can decrease extracellular ADP levels and increase adenosine levels. It is believed that these increased adenosine, binding to multiple receptors, exerts anti-inflammatory effects in the vasculature and dis-aggregatory effects on platelets, preventing further tissue damage at the wound site.

Additionally, it is believed by insight of the inventors, that activation of adenosine A2A receptors by adenosine (as a degradation product of eATP and eADP by Apyrase Agent and ubiquitous CD73) promotes collagen synthesis by human dermal fibrosis and increase the expression of vascular endothelial growth factor, basic fibroblast growth factor, and insulin-like growth factors which promote neovascularization (Valls M. D. et al. Adenosine receptor agonists for promotion of dermal wound healing. Biochemical Pharmacology. 77: 1117-1124, 2009). Adenosine also increases plasminogen activator release that plays a role in proteolytic degradation of extracellular matrices in tissue remodeling events required for normal repair of skin. It is believed by the inventor that Apyrase Agent's unexpected efficacy in treating tissue injury (alone or in combination with an antimicrobial) results from a clinically relevant increase in extracellular adenosine.

Taken together, Apyrase Agent contains appropriate absolute amounts and appropriate relative levels of APTase and ADPase activity to prevent accumulation of prothrombotic eADP, to more effectively generate anti-thrombotic and anti-inflammatory adenosine, and to promote neovasularization, when compared to other apyrases such as potato apyrase.

### Multidrug Resistance

Multidrug resistance can be caused by altered physiological states. eATP serves as “danger signal” that promotes biofilm formation or more antibiotic-resistant phenotypes of bacteria.

In addition, scavenging eATP re-sensitizes bacteria to antibiotic killing by preventing biofilm formation, maintaining susceptible physiological state, or compromising multidrug efflux pump efficiency.

### Sepsis

Sepsis causes cell activation and death that release massive eATP and eADP from the intracellular space to the extracellular space. eATP induces further inflammation and cell death, while eADP promotes development of microvascular thrombosis and release of entrophil extracellular traps from neutrophills. Hence, both eATP and eADP contribute to amplified systemic inflammation, microvascular thrombosis and ischemia, leading to multiple organ dysfunction.

Through insight of the inventor, while not limiting the scope of the instant invention, scavenging of eATP and eADP with Apyrase Agent will attenuate thrombosis and inflammation while generating anti-inflammatory adenosine. Meanwhile, Apyrase Agent will reduce bleeding complication by preventing platelet depletion and desensitization and maintaining vascular integrity. Together, Apyrase agent improves clinical outcomes and reduces mortality.

Apyrase Agent will be superior to potato apyrase to overcome sepsis-associated systemic inflammation and intravascular thrombosis as Apyrase Agent is far more stable at pH7.4 of human blood. Hence Apyrase Agent will be advantageous to reduce mortality and improve outcomes of sepsis patients.

### Heterotopic Ossification

It is believed, through insight of the inventor, that Apyrase Agent has efficacy in treating or preventing HO. Without being bound by theory, this surprising effectiveness may result, in part, to the ability of Apyrase Agent to cause a clinically significant decrease in eATP and eADP at a wound site and an increase in adenosine which inhibits inflammation at injury sites.

In HO, tissue injury can trigger inappropriate activation of mesenchymal stem cells (MSCs) along the osteogenic lineage, causing ectopic endochrondral heterotopic bone formation and functional contractures. Hydrolysis of eATP by Apyrase Agent generates adenosine that mitigates burn-induced BMP signaling and osteogenic differentiation in MSCs by increasing cAMP in MSCs at sites remote from burn injury.

Moreover, through insight of the inventor, Apyrase Agent is highly stable following topical administration due, in part, to post-translational modifications.

Apyrase Agent substantially overcomes the tendency for a subject to produce an immunologic response to non-human apyrase (e.g. potato apyrase) which can blunt a positive outcome. Hence Apyrase Agent will be advantageous to promote wound healing, prevent or eradicate bacterial infection in combination with antibiotics, and avoid HO formation without causing global osteopenia.

### EXAMPLES

The following examples are intended to illustrate but not to limit the invention. Moreover, scientific discussions below of underlying mechanisms gleaned from the data are also not meant as limitations of the inventions described here.

### Example 1

### Apyrase Agent Preparation

Apyrase Agent used in these examples is a soluble apyrase made from a construct coding for CD39L3 (e.g. Error! Reference source not found. of US 2015/0265685 A1), absent about 43 amino acids sequence from the N-terminus and absent about 44 amino acid sequence from the C-terminus corresponding to the membrane spanning domains as described by Jeong et al (U.S. Pat. No. 7,390,485). The Apyrase Agent further contains a substitution of an arginine for a glycine at residue 67 and substitution of a threonine for an arginine at residue 69 (where the residue number refers to the CD39L3 (Error! Reference source not found. of US 2015/0265685 A1).

Useful constructs are derived, in part, from Error! Reference source not found. of US 2015/0265685 A1 which codes for CD39L3.

The Apyrase Agent further contains a sequence encoding bovine α-lactalbumin signal peptide sequence.

The Apyrase Agent is transformed into a Chinese Hamster Ovary (CHO) cell lines by retrovector.

Conditioned medium is harvested from the transformed CHO cells and Apyrase Agent is purified by two-ion exchange chromatography steps (ANX and SP). Analysis of the N terminus of Apyrase Agent by this method reveals the following amino acids: Glu-Val-Leu-Pro-Pro-Gly-Leu-Lys-Tyr-Gly-Ile.

Additional details on methods of production of the Apyrase Agent used in these examples are found in U.S. Pat. No. 8,771,683.

### Example 2

### Studies to Demonstrate that Apyrase Agent Increases the Effectiveness of Antimicrobials

The effect of colistin, Apyrase agent, and combinations thereof were examined on E. coli growth.

E. coli strain K12 was cultivated to a concentration of about 0.1 OD (A_{600 nm).}

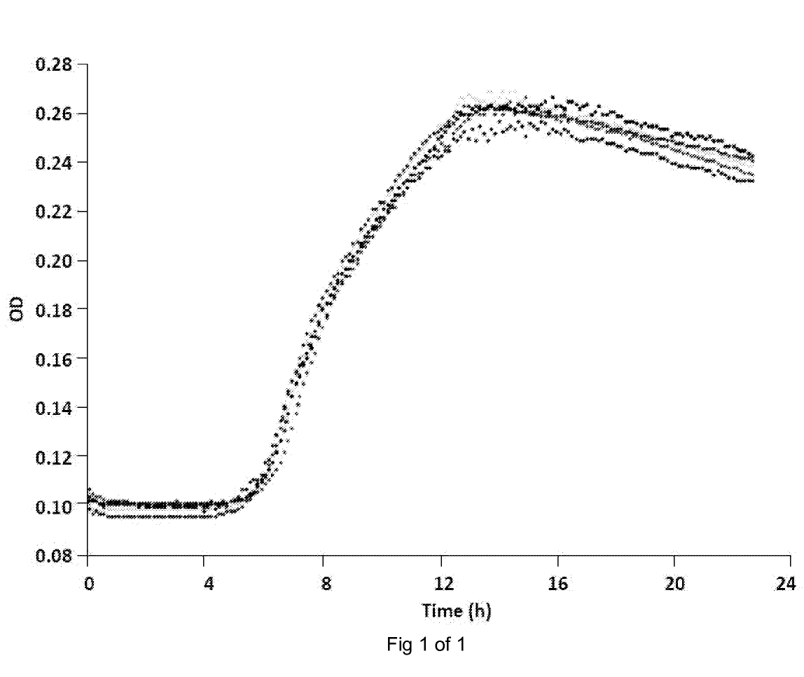

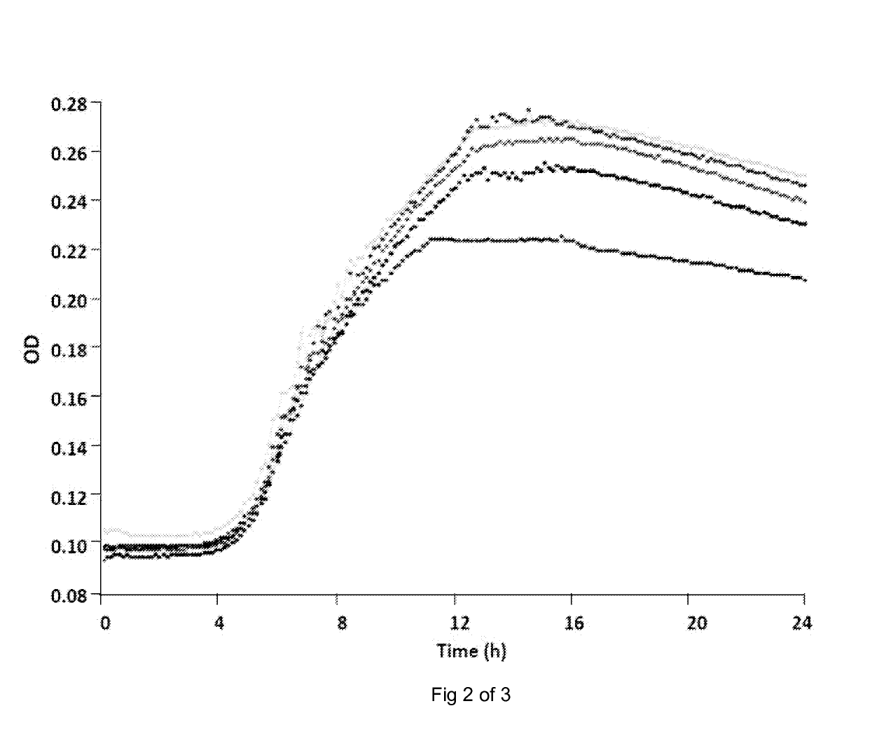

The effect of colistin on E. coli growth. Replicate cultures were cultivated in the presence of colistin (at a concentration of 0.25 μg/ml) and various amounts of additional Apyrase Agent buffer (Tris-biffered saline; 1, 3, 10, or 30 μL). As demonstrated in FIG. 1, colistin had no effect on E coli growth over a 24 hour period compared to, for example, the control of FIG. 2.

The effect of Apyrase Agent on E. coli growth. Replicate cultures were cultivated in the presence of Apyrase Agent at various concentrations (i.e. 1, 3, 10, or 30 U/ml). As shown in FIG. 2, Apyrase Agent had no inhibitory effect on E. coli growth at any concentration examined, when compared to control (bottom tracing).

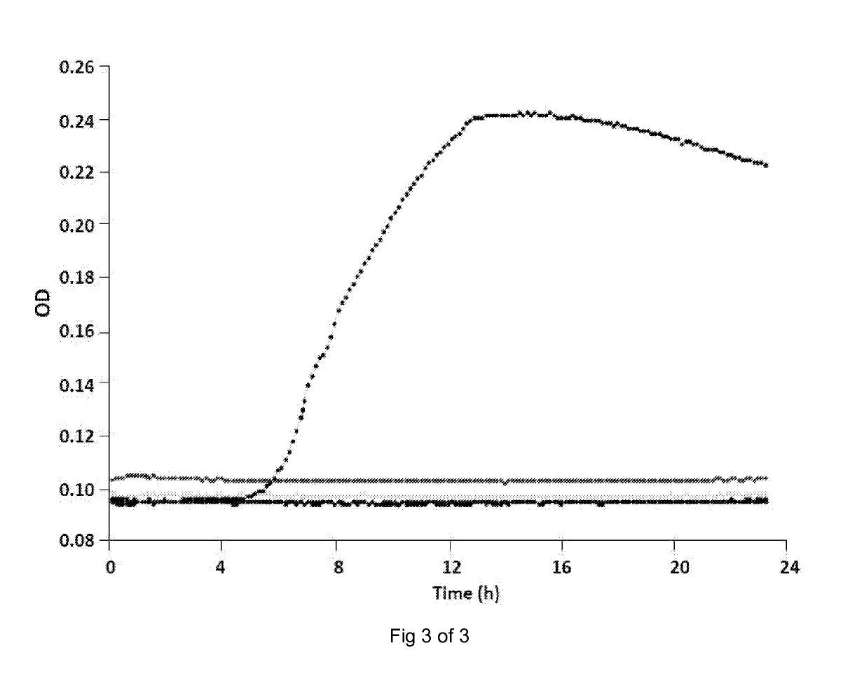

The effect of a combination of colistin and Apyrase Agent on E. coli growth. Replicate cultures were cultivated in the presence of colistin (at a concentration of 0.25 mg/ml) combined with Apyrase Agent at various concentrations (namely 1, 3, 10, or 30 U/ml). As shown in FIG. 3, colistin alone (upper tracing) had negligible effect on E. coli growth (compared to, for example, the control of FIG. 2). In contrast, colistin, when combined with Apyrase Agent had remarkable, synergistic inhibitory effect on E. coli growth.

Taken together, these results demonstrate the unexpected inhibitory effect of Apyrase Agent when combined with an antibiotic.

A similar study is performed substituting potato apyrase for Apyrase Agent. The results using Apyrase Agent are surprisingly superior to the results using potato apyrase.

### Example 3

### Studies to Demonstrate that Apyrase Agent Increases the Effectiveness of Antimicrobials

Approximately 10⁶ CFU/ml of A. baumannii strain ATCC 19606 is grown on solid medium culture plates in the presence of combinations of 0, 1, and 2 U/ml of Apyrase Agent and 0, 0.25, 0.5, 1, and 2 μg/ml of colistin. After incubation, the numbers of viable cells are determined by counting the colony forming units (CFU) on the plate (data provided as CFU/ml).

Apyrase Agent improves the efficiency of killing A. baumannii strain ATCC 19606 by colistin. 1 and 2 U/ml of Apyrase Agent reduces the viable cell count to below 10 to 100 CFU/ml while approximately 10⁵ CFU/ml remains in samples treated with colistin alone at the same concentrations.

In this Example (and in subsequent Examples), the colistin is colistin sulfate salt, catalog number C4461 from Sigma-Aldrich (St. Louis, Mo.) and the apyrase is catalog number M0393L from New England BioLabs Inc. (Ipswich, Mass.).

A similar study is performed substituting potato apyrase for Apyrase Agent. The results with Apyrase Agent are surprisingly superior to the results using potato apyrase.

### Example 4

### Studies to Demonstrate that Apyrase Agent Increases the Effectiveness of Antimicrobials

Approximately 10⁶ CFU/ml of A. baumannii strain DMCI is grown on solid medium culture plates in the presence of combinations of 0, 1, and 2 U/ml of Apyrase Agent and 0, 4, 8, 16, and 32 μg/ml of colistin. After incubation, the numbers of viable cells are determined by counting the colony forming units (CFU) on the plate (data provided as CFU/ml).

Apyrase Agent improves the efficiency of killing A. baumannii strain DMCI by colistin. 1 and 2 U/ml of Apyrase Agent reduces the viable cell count to below 10 to 100 CFU/ml while approximately 10⁷ CFU/ml remains in samples treated with colistin alone at the same concentrations.

A similar study is performed substituting potato apyrase for Apyrase Agent. The results with Apyrase Agent are surprisingly superior to the results using potato apyrase.

### Example 5

### Studies to Demonstrate that Apyrase Agent Increases the Effectiveness of Antimicrobials on an Antibiotic Sensitive Strain

Approximately 10⁵ to 10⁶ CFU/ml of colistin sensitive P. aeruginosa strain ATCC 27853 is grown in the presence of combinations of 0 and 2 U/ml of Apyrase Agent and 0, 0.25, and 0.5 μg/ml of colistin. After incubation, the numbers of viable cells are determined by counting the colony forming units (CFU) on the plate (data provided as CFU/ml). Apyrase Agent improves the efficiency of killing P. aeruginosa strain ATCC 27853 by colistin.

A similar study is performed substituting potato apyrase for Apyrase Agent. The results with Apyrase Agent are surprisingly superior to the results using potato apyrase.

### Example 6

### Studies to Demonstrate that Apyrase Agent Increases the Effectiveness of Antimicrobials

Approximately 10⁶ to 10⁷ CFU/ml of P. aeruginosa strain PAO300 is grown in the presence of combinations of 0 and 2 U/ml of Apyrase Agent and approximately 0 to 2 μg/ml of colistin. After incubation, the numbers of viable cells are determined by counting the colony forming units (CFU) on the plate (data provided as CFU/ml). Apyrase Agent improves the efficiency of killing P. aeruginosa strain PAO300 by colistin.

A similar study is performed substituting potato apyrase for Apyrase Agent. The results using Apyrase Agent are surprisingly superior to the results using potato apyrase.

### Example 7

### Studies to Demonstrate that Apyrase Agent Increases the Effectiveness of Antimicrobials Against Antibiotic Resistant Strains

Approximately 10⁵ to 10⁶ CFU/ml of the colistin-resistant A. baumannii strain VBA9 is grown in the presence of combinations of 0 and 2 U/ml of Apyrase Agent and approximately 0 to 32 μg/ml of colistin. After incubation, the numbers of viable cells are determined by counting the colony forming units (CFU) on the plate (data provided as CFU/ml). Apyrase Agent improves the efficiency of killing A. baumannii strain VBA9 by colistin. Apyrase Agent increases the effectiveness of colistin at less than 10-15 μg/ml such that less than 10 to 100 CFU/ml are detected relative to greater than 10⁶ to 10⁷ in the presence of colistin alone.

A similar study is performed substituting potato apyrase for Apyrase Agent. The results using Apyrase Agent are surprisingly superior to the results using potato apyrase.

### Example 8

### Studies to Demonstrate that Apyrase Agent Increases the Effectiveness of Antimicrobials Against Antibiotic Resistant Strains

Approximately 10⁵ to 10⁶ CFU/ml of the colistin-resistant A. baumannii strain AC285 is grown in the presence of combinations of 0 and 2 U/ml of Apyrase Agent and approximately 0 to 32 μg/ml of colistin. After incubation, the numbers of viable cells are determined by counting the colony forming units (CFU) on the plate (data provided as CFU/ml). Apyrase Agent improves the efficiency of killing A. baumannii strain AC285 by colistin. Apyrase Agent increases the effectiveness of colistin at less than 5-10 μg/ml such that less than 10 to 100 CFU/ml are detected relative to greater than 10⁷ to 10⁸ in the presence of colistin alone at the same concentrations.

A similar study is performed substituting potato apyrase for Apyrase Agent. The results using Apyrase Agent are surprisingly superior to the results using potato apyrase.

### Example 9

### Studies to Demonstrate that Apyrase Agent Increases the Effectiveness of Antimicrobials Against Antibiotic Resistant Strains

Approximately 10⁵ to 10⁶ CFU/ml of the colistin-resistant A. baumannii strain AC121 is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml). Apyrase Agent improves the efficiency of killing A. baumannii strain AC121 by colistin. Apyrase Agent increases the effectiveness of colistin at less than 20 μg/ml such that less than 10 to 100 CFU/ml are detected relative to greater than 10⁷ to 10⁸ in the presence of colistin alone at the same concentrations.

A similar study is performed substituting potato apyrase for Apyrase Agent. The results using Apyrase Agent are surprisingly superior to the results using potato apyrase.

### Example 10

### Studies to Demonstrate that Apyrase Agent Increases the Effectiveness of Antimicrobials

P. aeruginosa strain AU1292 is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml). Apyrase Agent improves the efficiency of killing P. aeruginosa strain AU1292 by colistin. Apyrase Agent increases the effectiveness of colistin at less than 20 μg/ml such that less than 10 to 100 CFU/ml are detected relative to greater than 10⁴ to 10⁵ in the presence of colistin alone at the same concentrations.

A similar study is performed substituting potato apyrase for Apyrase Agent. The results using Apyrase Agent are surprisingly superior to the results using potato apyrase.

### Example 11

### Studies to Demonstrate that Apyrase Agent Increases the Effectiveness of Antimicrobials

P. aeruginosa strain AU7443, mucoid is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml). Apyrase Agent improves the efficiency of killing P. aeruginosa strain AU7443, mucoid by colistin. Apyrase Agent increases the effectiveness of colistin at less than 20 μg/ml such that less than 10 to 100 CFU/ml are detected relative to greater than 10⁴ to 10⁵ in the presence of colistin alone at the same concentrations.

A similar study is performed substituting potato apyrase for Apyrase Agent. The results with Apyrase Agent are surprisingly superior to the results using potato apyrase.

### Example 12

### Studies to Demonstrate that Apyrase Agent Increases the Effectiveness of Antimicrobials

Burkholderia vietnamiensis AU 10214 is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml). Apyrase Agent improves the efficiency of killing Burkholderia vietnamiensis AU 10214 by colistin. Apyrase Agent increases the effectiveness of colistin at less than 20 μg/ml such that less than 10 to 100 CFU/ml are detected relative to greater than 10⁵ to 10⁶ in the presence of colistin alone at the same concentrations.

A similar study is performed substituting potato apyrase for Apyrase Agent. The results using Apyrase Agent are surprisingly superior to the results using potato apyrase.

### Example 13

### Studies to Demonstrate that Apyrase Agent Increases the Effectiveness of Antimicrobials

Burkholderia cepacia AU1114 is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml). Apyrase Agent improves the efficiency of killing Burkholderia cepacia AU1114 by colistin. Apyrase Agent increases the effectiveness of colistin at less than 20 μg/ml such that less than 10 to 100 CFU/ml are detected relative to greater than 10⁵ to 10⁶ in the presence of colistin alone at the same concentrations.

A similar study is performed substituting potato apyrase for Apyrase Agent. The results using Apyrase Agent are surprisingly superior to the results using potato apyrase.

### Example 14

### Studies to Demonstrate that Apyrase Agent Increases the Effectiveness of Antimicrobials

P. aeruginosa strain AU8104 is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml).

In the presence of colistin alone, greater than 105 to 106 CFU/ml are detected.

In the presence of colistin (at the same concentration) plus potato apyrase,t less than 10 to 100 CFU/ml are detected r.

In the presence of colistin (at the same concentration) plus Apyrase Agent, surprisingly superior efficiency of killing is observed when compared to colistin alone, or colistin plus potato apyrase.

### Example 15

### Studies to Demonstrate that Apyrase Agent Increases the Effectiveness of Antimicrobials

Burkholderia cepacia GUIa AU0019 is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml). Apyrase Agent improves the efficiency of killing Burkholderia cepacia GUIa AU0019 by colistin. Apyrase Agent increases the effectiveness of colistin at less than 10 μg/ml such that less than 10 to 100 CFU/ml are detected relative to greater than 10⁵ to 10⁶ in the presence of colistin alone at the same concentrations.

A similar study is performed substituting potato apyrase for Apyrase Agent. The results using Apyrase Agent are surprisingly superior to the results using potato apyrase.

### Example 16

### Studies to Demonstrate that Apyrase Agent Increases the Effectiveness of Antimicrobials

Burkholderia cepacia GIIIb AU0062 is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml). Apyrase Agent improves the efficiency of killing Burkholderia cepacia GIIIb AU0062 by colistin. Apyrase Agent increases the effectiveness of colistin at less than 20 μg/ml such that less than 10 to 100 CFU/ml are detected relative to greater than 10⁴ to 10⁵ in the presence of colistin alone at the same concentrations. Apyrase Agent is more effective than potato apyrase.

### Example 17

### Studies to Demonstrate that Apyrase Agent Increases the Effectiveness of Antimicrobials

Burkholderia ambifaria AU5203 is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml). Apyrase Agent improves the efficiency of killing Burkholderia ambifaria AU5203 by colistin. Apyrase Agent increases the effectiveness of colistin at less than 20 μg/ml such that less than 10 to 100 CFU/ml are detected relative to greater than 10⁵ to 10⁶ in the presence of colistin alone at the same concentrations.

A similar study is performed substituting potato apyrase for Apyrase Agent. The results using Apyrase Agent are surprisingly superior to the results using potato apyrase.

### Example 18

### Studies to Demonstrate that Apyrase Agent Increases the Effectiveness of Antimicrobials

Burkholderia cepacia GIIIb AU0055 is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml). Apyrase Agent improves the efficiency of killing Burkholderia cepacia GIIIb AU0055 by colistin. Apyrase Agent increases the effectiveness of colistin at less than 10 μg/ml such that less than 10 to 100 CFU/ml are detected relative to greater than 10⁴ to 10⁵ in the presence of colistin alone at the same concentrations.

A similar study is performed substituting potato apyrase for Apyrase Agent. The results using Apyrase Agent are surprisingly superior to the results using potato apyrase.

### Example 19

### Studies to Demonstrate that Apyrase Agent Increases the Effectiveness of Antimicrobials

P. aeruginosa AU0584 is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml). Apyrase Agent improves the efficiency of killing P. aeruginosa AU0584 by colistin. Apyrase Agent increases the effectiveness of colistin at less than 10 μg/ml such that less than 10 to 100 CFU/ml are detected relative to greater than 10³ to 10⁴ in the presence of colistin alone at the same concentrations.

A similar study is performed substituting potato apyrase for Apyrase Agent. The results using Apyrase Agent are surprisingly superior to the results using potato apyrase.

### Example 20

### Studies to Demonstrate that Apyrase Agent Increases the Effectiveness of Antimicrobials

Burkholderia dolosa AU0589 is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml). Apyrase Agent improves the efficiency of killing Burkholderia dolosa AU0589 by colistin. Apyrase Agent increases the effectiveness of colistin at less than 20 μg/ml such that less than 10 to 100 CFU/ml are detected relative to greater than 10⁶ to 10⁷ in the presence of colistin alone at the same concentrations.

A similar study is performed substituting potato apyrase for Apyrase Agent. The results using Apyrase Agent are surprisingly superior to the results using potato apyrase.

### Example 21

### Studies to Demonstrate that Apyrase Agent Increases the Effectiveness of Antimicrobials

Burkholderia multivorans AU0801 is grown in the presence of combinations of 0 and 2 U/ml of Apyrase Agent and approximately 0 to 20 μg/ml of colistin. After incubation, the numbers of viable cells are determined by counting the colony forming units (CFU) on the plate (data provided as CFU/ml). Apyrase Agent improves the efficiency of killing Burkholderia multivorans AU0801 by colistin. Apyrase Agent increases the effectiveness of colistin at less than 10 μg/ml such that less than 10 to 100 CFU/ml are detected relative to greater than 10⁵ to 10⁶ in the presence of colistin alone at the same concentrations.

A similar study is performed substituting potato apyrase for Apyrase Agent. The results using Apyrase Agent are surprisingly superior to the results using potato apyrase.

### Example 22

### Studies to Demonstrate that Apyrase Agent Increases the Effectiveness of Antimicrobials

Approximately 10⁶ CFU/ml of E. coli K12 is grown in the presence of combinations of 0 and 2 U/ml of Apyrase Agent and approximately 0 to 40 μM of antibacterial polymer E2. After incubation, the numbers of viable cells are determined by counting the colony forming units (CFU) on the plate (data provided as CFU/ml). Apyrase Agent improves the efficiency of killing E. coli K12 by E2. Apyrase Agent increases the effectiveness of E2 polymer at less than 15 μM such that less than 10 to 100 CFU/ml are detected relative to greater than 10⁷ to 10⁸ in the presence of E2 polymer alone at the same concentrations.

A similar study is performed substituting potato apyrase for Apyrase Agent. The results using Apyrase Agent are surprisingly superior to the results using potato apyrase.

### Example 23

### Studies to Demonstrate that Apyrase Agent Increases the Effectiveness of Antimicrobials

Approximately 10⁵ to 10⁶ CFU/ml of S. aureus ATCC 25923 is grown in the presence of combinations of 0 and 2 U/ml of Apyrase Agent and approximately 0 to 12 μM of antibacterial polymer E2. After incubation, the numbers of viable cells are determined by counting the colony forming units (CFU) on the plate (data provided as CFU/ml). Apyrase Agent improves the efficiency of killing S. aureus ATCC 25923 by polymer E2. Apyrase Agent increases the effectiveness of E2 polymer at less than 4 μM such that less than 10 to 100 CFU/ml are detected relative to greater than 10⁵ to 10⁶ in the presence of E2 polymer alone at the same concentrations.

A similar study is performed substituting potato apyrase for Apyrase Agent. The results using Apyrase Agent are surprisingly superior to the results using potato apyrase.

### Example 24

### Studies to Demonstrate that Apyrase Agent Increases the Effectiveness of Antimicrobials

Approximately 10⁴ to 10⁵ CFU/ml of Mycobacterium immunogenum ATCC 700505 is grown in 7H9 medium for 4 hours at 37° C. in the presence of combinations of 0 and 2 U/ml of Apyrase Agent and approximately 0 to 40 μM of antibacterial polymer E4. After incubation, the numbers of viable cells are determined by counting the colony forming units (CFU) on the plate (data provided as CFU/ml). Apyrase Agent improves the efficiency of killing Mycobacterium immunogenum ATCC 700505 by polymer E4. Apyrase Agent increases the effectiveness of E4 polymer at less than 10 μM such that less than 10 to 100 CFU/ml are detected relative to greater than 10⁴ to 10⁵ in the presence of E4 polymer alone at the same concentrations.

A similar study is performed substituting potato apyrase for Apyrase Agent. The results using Apyrase Agent are surprisingly superior to the results using potato apyrase.

### Example 25

### Studies to Demonstrate that Apyrase Agent and an Antimicrobial Act to Permeabilize the Cell Membranes of Bacterial Cells

Bacterial membrane permeabilization is conducted as described in Bourbon, et al., Analytical Biochemistry 381(2): 279-81 (2008), incorporated herein by reference. In short, the green fluorescent dye SYTOX is added to bacterial cells to measure membrane permeabilization. SYTOX fluorescence is low outside a cell and is substantially increased when it is in a cell interior. Thus, an increase in fluorescence represents an increase in permeability of the cell membrane. Using an E. coli culture of about 10⁷ CFU/ml, SYTOX was added to monitor permeability in the presence of Apyrase Agent at 2 U/ml and/or colistin at 0, 0.25, 0.5, 1, and 2 U/ml. The increase of SYTOX fluorescence indicates increased permeabilization of the bacterial membrane in the presence of colistin and Apyrase Agent.

A similar study is performed substituting potato apyrase for Apyrase Agent. The results using Apyrase Agent are surprisingly superior to the results using potato apyrase.

### Example 26

### Studies to Demonstrate that Unexpected Efficacy of the Combination of Apyrase Agent and an Antimicrobial In Vivo

This study uses a mouse model for wound infection and healing. The colistin-sensitive strain A. baumannii ATCC 17978 is not detected on skin 24 hours after a burn in the presence of Apyrase Agent and colistin. In the presence of potato apyraseor colistin alone, A. baumannii is detected at amounts from 10² to 10⁵ CFU/g of skin. In these experiments, colistin is used at 0.2 μg/ml. A similar study is performed substituting potato apyrase for Apyrase Agent. The results using Apyrase Agent are surprisingly superior to the results using potato apyrase.

### Example 27

### Studies to Demonstrate that Unexpected Efficacy of the Combination of Apyrase Agent and an Antimicrobial In Vivo

e.g., in a mouse model for wound infection and healing. The colistin-resistant strain A. baumannii ATCC DMCI is decreasingly detected on skin 24 hours, 48 hours, and 7 days after a burn in the presence of Apyrase Agent and colistin. After 7 days, A. baumannii ATCC DMCI is not detectable in the presence of Apyrase Agent and colistin. In the presence of Apyrase Agent or colistin alone, A. baumannii is detected at amounts from 10⁵ to 10⁸ CFU/g of skin throughout the test period over 7 days. In these experiments, colistin is used at 4 μg/ml.

A similar study is performed substituting potato apyrase for Apyrase Agent. The results using Apyrase Agent are surprisingly superior to the results using potato apyrase.

### Example 28

### Studies to Demonstrate that Unexpected Efficacy of the Combination of Apyrase Agent and an Antimicrobial In Vivo

e.g., in a mouse model for wound infection and healing. After a burn, A. baumannii ATCC DMCI is inoculated on skin. After 24 hours, the wounds are treated with Apyrase Agent alone, colistin alone, or Apyrase Agent and colistin in combination. Skin samples are collected after another 24 hours and the CFU/g skin of A baumannii ATCC DMCI is determined. A. baumannii ATCC DMCI is not detectable in the presence of Apyrase Agent and colistin 48 hours after the burn. In the presence of Apyrase Agent or colistin alone, A. baumannii is detected at amounts of approximately 10⁸ CFU/g of skin.

A similar study is performed substituting potato apyrase for Apyrase Agent. The results using Apyrase Agent are surprisingly superior to the results using potato apyrase.

### Example 29

### Studies to Demonstrate that Unexpected Efficacy Apyrase Agent to Prevent or Reduce Heterotopic Ossification

During the development of embodiments of the technology, the relationship of burn injury and heterotopic ossification are studied in vivo.

In particular, a heterotopic ossification model using an achilles tenotomy is used to demonstrate that burn injury significantly drives heterotopic bone formation at the tenotomy site. The data demonstrate that after an Achilles tenotomy, mice with a burn injury develop significantly more bone and develop bone at an earlier time point than non-burn control mice.

Multiple studies have demonstrated a link between acute inflammation and BMP-2 signaling, ultimately increasing bone formation. At the cellular level, research has shown that aberrant expression of BMPs in adipose-derived MSCs near the wound site stimulates osteogenic differentiation of multipotent cells. In support of this, data collected using the burn mouse model show an increase in osteogenic differentiation of MSCs after burn injury and a significant decrease of osteogenic differentiation of MSCs after burn injury and Apyrase Agent treatment. Alizarin red is used in to determine, quantitatively by colorimetry, the presence of calcific deposition by cells of an osteogenic lineage. As such, it is a marker of matrix mineralization, a crucial step towards the formation of calcified extracellular matrix associated with true bone. Thus, alizarin red staining indicates differentiation of cells to bone lineages.

Consequently, it is contemplated that decreasing inflammation at the burn site with Apyrase Agent—thus decreasing ATP concentrations, which leads to decreased MSC BMP-2 signaling and decreased osteogenic differentiation—alleviates heterotopic ossification at wound sites. During the development of embodiments of the technology provided, data were collected demonstrating that by decreasing the inflammation at the burn site with Apyrase Agent, the osteogenic capacity and level of BMP-2 signaling of the MSCs is indeed decreased.

Thus, in an effort to mitigate this osteogenic microenvironment created by acute inflammation, embodiments of the technology relate to treating (e.g., by topical application) wounds (e.g., a burn) to decrease inflammation by reducing ATP concentrations. The resulting decrease in inflammation at the wound or burn site with Apyrase Agent decreases the osteogenic capacity and level of BMP-2 signaling of the MSCs. As such, it is contemplated that applying Apyrase Agent to the burn site at the time of or after the burn injury and Achilles tenotomy prevents heterotopic bone formation by decreasing inflammation at the burn injury site. Accordingly, Apyrase Agent finds use to inhibit mesenchymal stem cell osteogenesis through Apyrase Agent mediated suppression of burn inflammation.

A similar study is performed substituting potato apyrase for Apyrase Agent. The results using Apyrase Agent are surprisingly superior to the results using potato apyrase.

### Example 30

### Studies to Demonstrate that the Combination of Apyrase Agent and Colistin have Unexpected Efficacy in the Treatment of Gram-Negative ESKAPE Pathogens

Data are collected from systematic tests to investigate bacterial killing with colistin and either potato apyrase or Apyrase Agent. The bacteria species individually tested are Enterococcus faecium, Staphylococcus aureus, Klebsiella pneumoniae, Acinetobacter baumannii, Pseudomonas aeruginosa and Enterobacter spp.—acronymically dubbed ‘the ESKAPE pathogens”.

The materials and methods include colistin (colistin sulfate salt, C4461, Sigma-Aldrich, St. Louis, Mo.), potato apyrase (M0393L, New England BioLabs Inc., Ipswich, Mass.), and Apyrase Agent (prepared according to the present invention).

Dose-kill curves for 22 strains (colistin sensitive or resistant ˜10⁶ CFU/ml) are generated after exposure to colistin at different concentrations by itself or in combination with potato apyrase or Apyrase Agent (2 U/ml) for 4 hours at 37° C.

An established mouse wound model is used for these tests. Female pathogen-free C57BL/6 mice (Harlan, Indianapolis, Ind.), 12 weeks old, weighing ˜20-25 grams are used in all experiments. The skin over the lumbrosacral and back region is clipped using a 35-W model 5-55E electrical clipper (Oyster-Golden A-S, Head no. 80, blade size 40) by using the method described by Ipaktchi et al. with slight modification. Two hundred microliters of 0.9% saline suspension of A. baumannii DMCI (I×10⁶ cells/ml), which is a highly colistin resistant clinical strain, is used for wound infection experiments. Viable bacterial numbers are determined 24 hours after burn. The Student t-test is used for statistical analysis.

Experiments are carried out in six groups (I) a control group, which contains neither apyrase nor colistin; (2) potato apyrase group, which contains potato apyrase (I U/ml); (3) colistin group, which contains colistin (4 μg/ml); (4) potato apyrase and colistin group, (5) Apyrase Agent group, (6) Apyrase Agent and colistin group.

The treatment is applied immediately after inoculation. After 24 hours, the mice are given lethal IP injections of pentobarbital (150 mg/kg) and skin samples are collected using a scalpel and scissors.

Data show that colistin group and the control group has the highest viable bacteria number recovered from burn sites 24 hours after burn and those two groups have no significant difference. Apyrase Agent group had significantly reduced viable bacteria number compared to control, colistin, or potato apyrase group. The combination group with Apyrase Agent has the lowest viable bacteria number in all six groups. This result demonstrates that Apyrase Agent alone has the ability to reduce bacterial infection when applied to wounds and exhibits a synergistic killing effect with colistin against bacteria that are resistant to colistin.

### Example 32

### Studies to Demonstrate that Apyrase Agent have Unexpected Efficacy in the Treatment of Sepsis

In this study, polymicrobial sepsis is induced by subjecting mice to cecal ligation and puncture (Csoka B et al. CD39 improves survival in microbial sepsis by attenuating systemic inflammation. FASEB. 2015, in press).

Mice are injected intraperitoneally 6 h or 12 h after the CLP operation with placebo, potato apyrase group (Sigma-Aldrich, A6410), or Apyrase agent. Endpoints include mortality; blood chemistry for liver and kidney functions (BUN, Creat, AST, ALT, ALK. Glu) and physiological parameters for heart function (heart rate, blood pressure) and lung function (oxygenation: PaO₂); blood samples for cytokine profiling.

Data show that the control group has the highest mortality of 70-100% by day 10, the potato apyrase group showed a somewhat improved mortality, and the Apyrase Agent group has the lowest mortality which is significantly lower than potato apyrase and control group. In addition, Apyrase agent group has improved organ function and reduced inflammation. The results using Apyrase Agent are surprisingly superior to the results using potato apyrase.

### Example 33

### Studies to Demonstrate that Apyrase Agent in combination with an Antimicrobial has Unexpected Efficacy in the Treatment of Sepsis

This study follows the same basic protocol of the previous Example, except each of the three groups (control, potato apyrase, and Apyrase Agent) further include an antimicrobial.

The results of this study show that Apyrase Agent in combination with an antimicrobial show substantial efficacy, unexpectedly greater than Apyrase Agent alone (or potato apyrase plus an antimicrobial).

All publications and patents mentioned in the above specification are herein incorporated by reference in their entirety for all purposes. Various modifications and variations of the described compositions, methods, and uses of the technology will be apparent to those skilled in the an without departing from the scope and spirit of the technology as described. Although the technology has been described in connection with specific exemplary embodiments, it should be understood that the technology as claimed should not be unduly limited to such specific embodiments. Indeed, various modifications of the described modes for carrying out the technology that are obvious to those skilled in pharmacology, biochemistry, medical science, or related fields are intended to be within the scope of the following claims.

1. A composition comprising and Apyrase Agent and an antimicrobial,

2. The composition of claim 1 wherein the antimicrobial is colistin.

3. The composition of claim 1 wherein the antimicrobial is an antibacterial polymer.

4. The composition of claim 3 wherein the antibacterial polymer is a polymer E2, a polymer E4, or a combination thereof.

5. The composition of claim 1 wherein the Apyrase Agent is present at a concentration of from about 1 to about 2 U/mL

6. The composition of claim 1 wherein the antimicrobial is selected from the group consisting of macrolides, penicillins, cephalosporins, carbepenems, monobactams, beta-lactam inhibitors, oxalines, aminoglycosides, chloramphenicols, sulfonamides, glycopeptides, quinolones, tetracyclines, fusidic acids, trimethoprims, metronidazoles, clindamycins, mupirocins, rifamycins, streptogramins, lipoproteins, polyenes, azoies, echinocandins, erythromycins, nafciilins, cefazolins, imipenems. aztreonams, gentamicins, sulfamethoxazoles, vancomycins, ciprofloxacins, rifampins, teicopianins, azithromycins, clarithromycins, ofloxacins, lomefloxacins, norfloxacins, nalidixic acids, sparfloxacins, pefloxacins, amifloxacins, gatifloxacins, moxifloxacins, gemifloxacins, enoxacins, fieroxacins, minocyclines, linezolids, temafloxacins, tosufloxacins, ciinafloxacins, sulbactams, clavuianic acids, amphotericin Bs, fluconazoles, itraconazoies, ketoconazoles, and nystatins.

7. The composition of claim 1 further comprising a dressing or a bandage.

8. The composition of claim 1 wherein the composition is a gel, an ointment, a solution, a cream, a salve, or a spray.

9. A method for treating or preventing an infection or colonization in a subject, the method comprising:
a) identifying a subject in need of an antimicrobial treatment; and
b) administering to the subject an Apyrase Agent and an antimicrobial.

10. The method of claim 9 wherein the Apyrase Agent and the antimicrobial are administered sequentially.

11. The method of claim 9 wherein the Apyrase Agent and the antimicrobial are administered simultaneously.

12. The method of claim 9 wherein the antimicrobial is colistin, polymer E2, polymer E4, or a combination thereof.

13. The method of claim 9 wherein the antimicrobial belongs to a class selected from the group consisting of macrolide, penicillin, cephalosporin, carbepenem, monobactam, beta-lactam inhibitor, oxaiine, aminoglycoside, chloramphenicol, sulfonamide, glycopeptides, quinolone, tetracycline, fusidic acid, trimethoprim, metronidazole, clindamycin, mupirocin, rifamycins, streptogramin, lipoprotein, polyene, azoie, and echinocandin.

14. The method of claim 9 wherein the antimicrobial is selected from the group consisting of erythromycin, nafcillin, cefazolin, imipenem, aztreonam, gentamicin, sulfamethoxazole, vancomycin, ciprofloxacin, trimethoprim, rifampin, metronidazole, clindamycin, teicopianin, mupirocin, azithromycin, clarithromycin, ofloxacin, lomefioxacin, norfloxacin, nalidixic acid, sparfioxacin, pefloxacin, amifloxacin, gatifloxacin, moxifloxacin, gemifloxacin, enoxacin, fieroxacin, minocycline, iinezolid, femafloxacin, tosufloxacin, clinafloxacin, sulbactam, ciavulanic acid, amphotericin B, fluconazole, itraconazole, ketoconazole, and nystatin.

15. The method of claim 9 further comprising testing the subject for an infection and/or colonization.

16. The method of claim 15 wherein the testing is performed before the administering.

17. The method of claim 15 wherein the testing is after the administering.

18. The method of claim 15 further comprising a second administering to the subject of the Apyrase Agent and the antimicrobial.

19. The method of claim 18 wherein a dosage of the second administering is determined based on a result of the testing.

20. The method of claim 9 wherein the administering occurs by a route selected from the group consisting of oral intravenous, intraperitoneal, intramuscular, transdermal, subcutaneous, topical, sublingual, and rectal.

21-33. (canceled)
